SEQUENCE LISTING (37 sequences)

SEQ ID NO: 1 - protein, 122 aa, Homo sapiens
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 2 - protein, 122 aa, Homo sapiens
MKLLTGLVFCSLVLGVSSRSFFSFLGEAFDGARDMWRAYSDMREANYIGSDKYFHARGNYDAAKRGPGGAWAAEVISDARENIQRFFGHGAEDSLADQAANEWGRSGKDPNHFRPAGLPEKY

SEQ ID NO: 3 - protein, 104 aa, Homo sapiens
RSFFSFLGEAFDGARDMWRAYSDMREANYIGSDKYFHARGNYDAAKRGPGGAWAAEVISDARENIQRFFGHGAEDSLADQAANEWGRSGKDPNHFRPAGLPEKY

SEQ ID NO: 4 - protein, 122 aa, Homo sapiens
MKLLTGLVFCSLVLSVSSRSFFSFLGEAFDGARDMWRAYSDMREANYIGSDKYFHARGNYDAAKRGPGGAWAAEVISNARENIQRLTGRGAEDSLADQAANKWGRSGRDPNHFRPAGLPEKY

SEQ ID NO: 5 - protein, 104 aa, Homo sapiens
RSFFSFLGEAFDGARDMWRAYSDMREANYIGSDKYFHARGNYDAAKRGPGGAWAAEVISNARENIQRLTGRGAEDSLADQAANKWGRSGRDPNHFRPAGLPEKY

SEQ ID NO: 6 - protein, 83 aa, Homo sapiens
MKLLTGLVFCSLVLSVSSRSFFSFLGEAFDGARDMWRAYSDMREANYIGSDKYFHARGNYDAAKRGPGGAWAAEVISLFSAEL

SEQ ID NO: 7 - protein, 65 aa, Homo sapiens
RSFFSFLGEAFDGARDMWRAYSDMREANYIGSDKYFHARGNYDAAKRGPGGAWAAEVISLFSAEL

SEQ ID NO: 8 - protein, 130 aa, Homo sapiens
MRLFTGIVFCSLVMGVTSESWRSFFKEALQGVGDMGRAYWDIMISNHQNSNRYLYARGNYDAAQRGPGGVWAAKLISRSRVYLQGLIDCYLFGNSSTVLEDSKSNEKAEEWGRSGKDPDRFRPDGLPKKY

SEQ ID NO: 9 - protein, 112 aa, Homo sapiens
ESWRSFFKEALQGVGDMGRAYWDIMISNHQNSNRYLYARGNYDAAQRGPGGVWAAKLISRSRVYLQGLIDCYLFGNSSTVLEDSKSNEKAEEWGRSGKDPDRFRPDGLPKKY

SEQ ID NO: 10 - protein, 93 aa, Homo sapiens
MDRLQTALLVVLVLLAVALQATEAGPYGANMEDSVCCRDYVRYRLPLRVVKHFYWTSDSCPRPGVVLLTFRDKEICADPRVPWVKMILNKLSQ

SEQ ID NO: 11 - protein, 69 aa, Homo sapiens
GPYGANMEDSVCCRDYVRYRLPLRVVKHFYWTSDSCPRPGVVLLTFRDKEICADPRVPWVKMILNKLSQ

SEQ ID NO: 12 - protein, 38 aa, Homo sapiens
ESVTVTRDLEGTYLCRARSTQGEVTREPPGMRLSSSLW

SEQ ID NO: 13 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 14 - protein, 206 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 15 - protein, 232 aa, Homo sapiens; identical to SEQ ID NO: 13

SEQ ID NO: 16 - protein, 215 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 17 - protein, 209 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 18 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 19 - protein, 174 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 20 - protein, 171 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 21 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 22 - protein, 147 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 23 - protein, 137 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRCDKPRR

SEQ ID NO: 24 - protein, 371 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 25 - protein, 327 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 26 - protein, 395 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 27 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 28 - protein, 371 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 29 - protein, 389 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 30 - protein, 354 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 31 - protein, 317 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRCDKPRR

SEQ ID NO: 32 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 33 - protein, 188 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDSPRPLCPRCTQHHQRPDPRTCRCRCRRRSFLRCQGRGLELNPDTCRCRKLRR

SEQ ID NO: 34 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 35 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 36 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 37 - protein, 77 aa, Homo sapiens
AVLPRSAKELRCQCIKTYSKPFHPKFIKELRVIESGPHCANTEIIVKLSDGRELCLDPKENWVQRVVEKFLKRAENS

(19) United States
(12) Patent Application Publication
(10) Pub. No.: US 2019/0346459 A1
(43) Pub. Date: Nov. 14, 2019

(54) BIOMARKERS OF BLOOD-BRAIN BARRIER DYSFUNCTION

(71) Applicants: NESTEC S.A., Vevey (CH); CHUV, Lausanne (CH)

(72) Inventors: Gene Bowman, St-Sulpice (CH); Loic Dayon, Colombier (CH); Eugenia Migliavacca, Lausanne (CH); Julius Popp, Lausanne (CH)

(21) Appl. No.: 16/312,355

(22) PCT Filed: Jun. 22, 2017
(86) PCT No.: PCT/EP2017/065340

§ 371 Date: Dec. 21, 2018

(30) Foreign Application Priority Data
Jun. 28, 2016 (EP) 16176659.7

(51) Int. Cl. G01N 33/68; A61K 31/202; A61K 31/4415; A61K 31/519; A61K 31/714

(52) U.S. Cl. CPC G01N 33/6896; A61K 31/202; A61K 31/4415; A61K 31/519; G01N 2800/2835; G01N 2333/4709; G01N 2333/5421; G01N 2333/70525; G01N 2800/2814; A61K 31/714

(57) ABSTRACT

A method for determining whether a subject has an impaired blood-brain barrier (BBB) or is at risk of developing an impaired blood-brain barrier (BBB) comprising determining the level of one or more biomarkers in one or more samples obtained from the subject, wherein the one or more biomarkers comprise serum amyloid A (SAA).

### FIELD OF THE INVENTION

The present invention relates to biomarkers and biomarker combinations that may be used to determine whether a subject has or is at risk of developing an impaired blood-brain barrier (BBB). The invention also relates to biomarkers and biomarker combinations that may be used to determine whether a subject is at risk of developing a cognitive impairment, for example Alzheimer's disease or vascular cognitive impairment.

### BACKGROUND TO THE INVENTION

The blood-brain barrier (BBB) is a selective barrier that separates circulating blood from the brain. The BBB is comprised of endothelial cells bound together by tight junction proteins that form the blood facing side of the lumen of the small cerebral blood vessels. In addition, astrocytes (in particular, projections from those cells termed astrocytic feet) and pericytes contribute to the structure and function of the BBB.

The endothelial cells of the BBB express multiple substrate-specific transport systems that control the transport of nutrients, energy metabolites, and other essential molecules from the blood into the brain and the transport of metabolic waste products from the brains interstitial fluid into the blood (Aspelund A. et al. 2015, J. Exp. Med 212, 991-999). Therefore, the BBB serves as a key homeostatic site of the nervous system since it connects the central nervous system (CNS) systemic circulation, and major systems in the body such as respiratory, renal, hepatic and immune systems (Zhao, Z et al. 2015, Cell 163, 1064-1078).

A number of studies have associated BBB dysfunction with cognitive impairment. For example, post-mortem analyses have demonstrated BBB damage in Alzheimer's disease patients (Zlokovic, B V, 2008, Neuron, 57, 178-201). In addition, neuroimaging studies have shown the accumulation of iron and microbleeds in Alzheimer's disease patients, which suggests subtle haemorrhage or rupture of small vessels in the brain at some point in life (Montagne A. et al. 2015, Neuron, 85, 296-302). Further studies have shown that cerebrospinal fluid (CSF)-to-serum ratios of blood-derived albumin are higher in all dementia patients (including those suffering from Alzheimer's disease) when compared against age-matched controls (Bowman G L et al. 2008 Aging Health, 4, 47-55). Indeed, this measure of BBB function associates with accelerated Alzheimer's disease progression independent of age and other Alzheimer's disease risk factors (Bowman G L et al., 2008, Neurology, 68, 1809-1814).

BBB dysfunction is considered a vascular contribution to the risk for the development of age-related cognitive decline, cognitive impairment and dementia, including Alzheimer's disease and its progression.

Accordingly, there exists a significant need for methods of identifying BBB dysfunction in living subjects, in particular in subjects that do not exhibit symptoms of or have not been diagnosed with a cognitive impairment. Early diagnosis of subjects with an impaired BBB may enable therapeutic intervention, which may prevent or reduce the risk of the subject developing conditions associated with an impaired BBB, for example cognitive impairments such as Alzheimer's disease (AD), mild cognitive impairment (MCI), vascular cognitive impairment, vascular dementia, Parkinson's disease (PD), traumatic brain injury (TBI), and age-related cognitive decline.

### SUMMARY OF THE INVENTION

The inventors have demonstrated that certain cerebrospinal fluid (CSF) and serum biomarkers can identify subjects, in particular older adults, with blood-brain barrier (BBB) impairment.

Specifically, the inventors collected CSF and serum samples from 118 adults aged 55 and older to analyse the cross-sectional relationship between biomarkers of inflammation and BBB function. BBB dysfunction was defined a priori as a CSF-to-serum albumin ratio greater than or equal to 9.0. The inventors carried out Least Absolute Shrinkage and Selection Operator (LASSO) logistic regression analysis to select the biomarkers that best classified subjects with BBB impairment. Subsequently, diagnostic accuracy was assessed by calculating area under the receiver operating characteristic (ROC) curve.

The inventors determined biomarkers for identifying BBB impairment. Such biomarkers include serum amyloid A (SAA), macrophage-derived chemokine (MDC; also known as C-C motif chemokine 22, CCL22), soluble inter-cellular adhesion molecule-1 (sICAM-1), vascular endothelial growth factor (VEGF) and/or interleukin 8 (IL-8).

Accordingly, in one aspect the invention provides a method for determining whether a subject has an impaired blood-brain barrier (BBB) or is at risk of developing an impaired blood-brain barrier (BBB) by comprising determining the level of one or more biomarkers in one or more samples obtained from the subject, wherein the one or more biomarkers comprise serum amyloid A (SAA).

In another aspect, the invention provides a method for determining whether a subject is at risk of developing a cognitive impairment by comprising determining the level of one or more biomarkers in one or more samples obtained from the subject, wherein the one or more biomarkers comprise serum amyloid A (SAA).

In one embodiment, the cognitive impairment is selected from the group consisting of Alzheimer's disease (AD), vascular cognitive impairment and vascular dementia, Parkinson's disease (PD), age-related cognitive decline, and traumatic brain injury (TBI). Preferably, the cognitive impairment is Alzheimer's disease.

In one embodiment, the SAA is human SAA.

In one embodiment, the SAA is SAA1, SAA2 or SAA4, preferably SAA1.

In one embodiment, the method further comprises determining the level of macrophage-derived chemokine (MDC) in a sample from the subject.

In one embodiment, the method further comprises determining the level of one or more biomarkers selected from the group consisting of soluble inter-cellular adhesion molecule-1 (sICAM-1), vascular endothelial growth factor (VEGF) and interleukin 8 (IL-8), in one or more samples obtained from the subject.

In one embodiment, the method further comprises determining the level of soluble inter-cellular adhesion molecule-1 (sICAM-1) in a sample from the subject.

In one embodiment, the method further comprises determining the level of vascular endothelial growth factor (VEGF) in a sample from the subject.

In one embodiment, the method further comprises determining the level of interleukin 8 (IL-8) in a sample from the subject.

In another aspect, the invention provides a method for determining whether a subject has an impaired blood-brain barrier (BBB) or is at risk of developing an impaired blood-brain barrier (BBB) comprising determining the level of one or more biomarkers in one or more samples obtained from the subject, wherein the one or more biomarkers comprise macrophage-derived chemokine (MDC). The method may further comprise determining the level of one or more biomarkers selected from the group consisting of serum amyloid A (SAA), soluble inter-cellular adhesion molecule-1 (sICAM-1), vascular endothelial growth factor (VEGF) and interleukin 8 (IL-8), in one or more samples obtained from the subject.

In another aspect, the invention provides a method for determining whether a subject is at risk of developing a cognitive impairment comprising determining the level of one or more biomarkers in one or more samples obtained from the subject, wherein the one or more biomarkers comprise macrophage-derived chemokine (MDC). The method may further comprise determining the level of one or more biomarkers selected from the group consisting of serum amyloid A (SAA), soluble inter-cellular adhesion molecule-1 (sICAM-1), vascular endothelial growth factor (VEGF) and interleukin 8 (IL-8), in one or more samples obtained from the subject.

In another aspect, the invention provides a method for determining whether a subject has an impaired blood-brain barrier (BBB) or is at risk of developing an impaired blood-brain barrier (BBB) comprising determining the level of one or more biomarkers in one or more samples obtained from the subject, wherein the one or more biomarkers comprise soluble inter-cellular adhesion molecule-1 (sICAM-1). The method may further comprise determining the level of one or more biomarkers selected from the group consisting of serum amyloid A (SAA), macrophage-derived chemokine (MDC), vascular endothelial growth factor (VEGF) and interleukin 8 (IL-8), in one or more samples obtained from the subject.

In another aspect, the invention provides a method for determining whether a subject is at risk of developing a cognitive impairment comprising determining the level of one or more biomarkers in one or more samples obtained from the subject, wherein the one or more biomarkers comprise soluble inter-cellular adhesion molecule-1 (sICAM-1). The method may further comprise determining the level of one or more biomarkers selected from the group consisting of serum amyloid A (SAA), macrophage-derived chemokine (MDC), vascular endothelial growth factor (VEGF) and interleukin 8 (IL-8), in one or more samples obtained from the subject.

In another aspect, the invention provides a method for determining whether a subject has an impaired blood-brain barrier (BBB) or is at risk of developing an impaired blood-brain barrier (BBB) comprising determining the level of one or more biomarkers in one or more samples obtained from the subject, wherein the one or more biomarkers comprise vascular endothelial growth factor (VEGF). The method may further comprise determining the level of one or more biomarkers selected from the group consisting of serum amyloid A (SAA), macrophage-derived chemokine (MDC), soluble inter-cellular adhesion molecule-1 (sICAM-1) and interleukin 8 (IL-8), in one or more samples obtained from the subject.

In another aspect, the invention provides a method for determining whether a subject is at risk of developing a cognitive impairment comprising determining the level of one or more biomarkers in one or more samples obtained from the subject, wherein the one or more biomarkers comprise vascular endothelial growth factor (VEGF). The method may further comprise determining the level of one or more biomarkers selected from the group consisting of serum amyloid A (SAA), macrophage-derived chemokine (MDC), soluble inter-cellular adhesion molecule-1 (sICAM-1) and interleukin 8 (IL-8), in one or more samples obtained from the subject.

In another aspect, the invention provides a method for determining whether a subject has an impaired blood-brain barrier (BBB) or is at risk of developing an impaired blood-brain barrier (BBB) comprising determining the level of one or more biomarkers in one or more samples obtained from the subject, wherein the one or more biomarkers comprise interleukin 8 (IL-8). The method may further comprise determining the level of one or more biomarkers selected from the group consisting of serum amyloid A (SAA), macrophage-derived chemokine (MDC), soluble inter-cellular adhesion molecule-1 (sICAM-1) and vascular endothelial growth factor (VEGF), in one or more samples obtained from the subject.

In another aspect, the invention provides a method for determining whether a subject is at risk of developing a cognitive impairment comprising determining the level of one or more biomarkers in one or more samples obtained from the subject, wherein the one or more biomarkers comprise interleukin 8 (IL-8). The method may further comprise determining the level of one or more biomarkers selected from the group consisting of serum amyloid A (SAA), macrophage-derived chemokine (MDC), soluble inter-cellular adhesion molecule-1 (sICAM-1) and vascular endothelial growth factor (VEGF), in one or more samples obtained from the subject.

In one embodiment, the method comprises determining the level of SAA, MDC, sICAM-1, VEGF and IL-8 in one or more samples obtained from the subject.

In one embodiment, the level of the one or more biomarkers is compared with one or more reference values. In this case, preferably each biomarker level in each sample and the corresponding reference values are determined using the same analytical method. The reference values may be based on values (e.g. averages) of the one or more biomarkers in populations of subjects who have, for example, previously been identified as having normal or impaired blood-brain barriers.

In one embodiment, the method further comprises combining the level of the one or more biomarkers with one or more demographic, clinical and/or lifestyle characteristics of the subject. Preferably, the demographic variables include age, gender and education level.

Preferably the clinical variables include the presence of other disease conditions such as diabetes, obesity and hypertension. Preferably, the lifestyle characteristic is whether the subject is a smoker or a non-smoker.

In another embodiment, the method further comprises combining the level of the one or more biomarkers with one or more demographic, clinical and/or lifestyle characteristics of the subject wherein the clinical measures include Alzheimer's disease biological parameters such as the presence of ApoEe4 allele, Clinical Dementia Rating (CDR), CSF abeta1-42, phospho-tau181 and total tau (t-tau).

In one embodiment, the method further comprises combining the level of the one or more biomarkers with the gender of the subject.

In one embodiment, the method further comprises combining the level of the one or more biomarkers with the age of the subject.

In one embodiment, the method comprises determining a value that represents the prediction of blood-brain barrier impairment (BBB). This may be termed a blood-brain barrier impairment score (S) and may be calculated using the formula:

S=A+B×(IL-8)+C×(MDC)+D×(SAA)+E×(sICAM-1)+F×(VEGF)+G×(Gender)

wherein A, B, C, D, E, F and G are coefficients. The coefficients may be chosen based on a pre-determined model. Blood-brain barrier impairment may be predicted if S is above or below a pre-determined level, for example if S>0.

In one embodiment, the method comprises determining a blood-brain barrier impairment score (S) using the formula:

$$S = - 1.04 + 6.20 \times {10}^{- 4} \times {log}_{10} ( IL \text{-} 8 ) + 2.24 \times {10}^{- 1} \times {log}_{10} ( MDC ) + 2.33 \times {10}^{- 1} \times {log}_{10} ( SAA ) + 1.28 \times {log}_{10} ( sICAM \text{-} 1 ) + 4.31 \times {10}^{- 1} \times {log}_{10} ( VEGF ) - 5.16 \times {10}^{- 1} \times SEX\_VALUE$$

wherein SEX_VALUE=−sqrt(2)/2 for males and +sqrt(2)/2 for females represents the gender, and wherein blood-brain barrier (BBB) impairment is predicted if S>0. Biomarkers sICAM-1, VEGF, IL-8, SAA and MDC are measured in pg/mL.

In one embodiment, the level of SAA is determined in a serum sample. In another embodiment, the level of SAA is determined in a cerebrospinal fluid (CSF) sample.

In one embodiment, the level of MDC is determined in a serum sample. In another embodiment, the level of MDC is determined in a cerebrospinal fluid (CSF) sample.

In one embodiment, the level of sICAM-1 is determined in a serum sample. In another embodiment, the level of sICAM-1 is determined in a cerebrospinal fluid (CSF) sample.

In one embodiment, the level of VEGF is determined in a serum sample. In another embodiment, the level of VEGF is determined in a cerebrospinal fluid (CSF) sample.

In one embodiment, the level of IL-8 is determined in a serum sample. In another embodiment, the level of IL-8 is determined in a cerebrospinal fluid (CSF) sample.

In one embodiment, the levels of the one or more biomarkers are determined in one or more CSF samples.

In one embodiment, the subject is a human.

In one embodiment, the subject is an ageing human. In another embodiment, the subject is a human over the age of 30, 35, 40, 45, 50, 55, 60, 65, 70, 75, 80, 85, 90, 95 or 100 years old. Preferably, the subject is a human over the age of 55 years old.

In one embodiment, the subject substantially does not exhibit any symptoms of a cognitive impairment.

In one embodiment, the subject has not been diagnosed with a cognitive impairment.

Preferably, the method is an in vitro method.

In another aspect, the invention provides a kit for determining whether a subject has an impaired blood-brain barrier (BBB) or is at risk of developing an impaired blood-brain barrier (BBB), wherein the kit comprises one or more antibodies, preferably 2, 3, 4 or 5 antibodies, wherein each antibody is specific for a biomarker as disclosed herein.

In another aspect, the invention provides a kit for determining whether a subject is at risk of developing a cognitive impairment, wherein the kit comprises one or more antibodies, preferably 2, 3, 4 or 5 antibodies, wherein each antibody is specific for a biomarker as disclosed herein.

In another aspect, the invention provides a method of treating or preventing blood-brain barrier (BBB) impairment comprising the steps:
(a) determining whether a subject has an impaired blood-brain barrier (BBB) or is at risk of developing an impaired blood-brain barrier (BBB) according to the method of the invention; and
(b) applying an intervention capable of improving blood-brain barrier (BBB) function to a subject identified to be in need thereof.

In another aspect, the invention provides a method of preventing or reducing the risk of a cognitive impairment comprising the steps:
(a) determining whether a subject is at risk of developing a cognitive impairment according to the method of the invention; and
(b) applying an intervention capable of preventing or reducing the risk of a cognitive impairment to a subject identified to be in need thereof.

In one embodiment, the intervention is a dietary intervention.

In one embodiment, the dietary intervention comprises increasing vitamin B intake by the subject, preferably by administering a vitamin B supplement.

In one embodiment, the dietary intervention comprises increasing omega-3 fatty acid intake by the subject, preferably by administering an omega-3 fatty acid supplement.

In another aspect, the invention provides a method of selecting a modification in lifestyle of a subject comprising the steps:
(a) determining whether the subject has an impaired blood-brain barrier (BBB) or is at risk of developing an impaired blood-brain barrier (BBB) according to the method of the invention; and
(b) selecting a modification in lifestyle capable of improving blood-brain barrier (BBB) function in a subject identified to be in need thereof.

In another aspect, the invention provides a method of selecting a modification in lifestyle of a subject comprising the steps:
(a) determining whether the subject is at risk of developing a cognitive impairment according to the method of the invention; and
(b) selecting a modification in lifestyle capable of preventing or reducing the risk of a cognitive impairment in a subject identified to be in need thereof.

In one embodiment, the method further comprises applying the selected modification in lifestyle to the subject.

In one embodiment, the modification in lifestyle comprises a dietary intervention as disclosed herein.

In another aspect, the invention provides a diet product for use in treating or preventing blood-brain barrier (BBB) impairment, wherein the diet product is administered to a subject determined to have an impaired blood-brain barrier or to be at risk of developing an impaired blood-brain barrier (BBB) according to the method of the invention.

In another aspect, the invention provides a diet product for use in preventing or reducing the risk of a cognitive impairment, wherein the diet product is administered to a subject determined to be at risk of developing a cognitive impairment according to the method of the invention.

In another aspect, the invention provides the use of a diet product for the manufacture of a medicament for treating or preventing blood-brain barrier (BBB) impairment, wherein the diet product is administered to a subject determined to have an impaired blood-brain barrier (BBB) or to be at risk of developing an impaired blood-brain barrier (BBB) according to the method of the invention.

In another aspect, the invention provides the use of a diet product for the manufacture of a medicament for preventing or reducing the risk of a cognitive impairment, wherein the diet product is administered to a subject determined to be at risk of developing a cognitive impairment according to the method of the invention.

In another aspect, the invention provides the use of a diet product for treating or preventing blood-brain barrier (BBB) impairment, wherein the diet product is administered to a subject determined to have an impaired blood-brain barrier (BBB) or to be at risk of developing an impaired blood-brain barrier (BBB) according to the method of the invention.

In another aspect, the invention provides the use of a diet product for preventing or reducing the risk of a cognitive impairment, wherein the diet product is administered to a subject determined to be at risk of developing a cognitive impairment according to the method of the invention.

In one embodiment, the diet product is a vitamin B supplement. In another embodiment, the diet product is an omega-3 fatty acid supplement.

In another aspect, the invention provides a computer program product comprising computer implementable instructions for causing a programmable computer to determine whether a subject has an impaired blood-brain barrier (BBB) or is at risk of developing an impaired blood-brain barrier (BBB) according to the method disclosed herein.

In another aspect, the invention provides a computer program product comprising computer implementable instructions for causing a programmable computer to determine whether a subject is at risk of developing a cognitive impairment according to the method disclosed herein.

In another aspect, the invention provides a computer program product comprising computer implementable instructions for causing a programmable computer to determine whether a subject has an impaired blood-brain barrier (BBB) or is at risk of developing an impaired blood-brain barrier (BBB) given the levels of one or more biomarkers from the user, wherein the biomarkers are selected from the one or more biomarkers as disclosed herein.

In another aspect, the invention provides a computer program product comprising computer implementable instructions for causing a programmable computer to determine whether a subject is at risk of developing a cognitive impairment given the levels of one or more biomarkers from the user, wherein the biomarkers are selected from the one or more biomarkers as disclosed herein.

### DESCRIPTION OF THE DRAWINGS

FIG. 1

Cerebrospinal fluid (CSF) inflammatory signature of blood-brain barrier (BBB) impairment in older adults. Receiver operating characteristic (ROC) curves for diagnosis of blood-brain barrier (BBB) impairment for Reference (labelled “REF ref”) and Best models (labelled “neuro”). For the Reference model the area under the curve (AUC) is 0.80 whereas for the Best model the area under the curve (AUC) is 0.95. The variables selected in the Best model are: Gender and 5 CSF biomarkers (IL-8, sICAM-1, VEGF, SAA, and MDC).

FIG. 2

Correlation in the concentrations of serum amyloid A (SAA) measured both in CSF and serum in the cohort under study. Concentrations (in pg/mL) are log-transformed. R is 0.7083, R2 is 0.5017 and p<1 e-5.

### DETAILED DESCRIPTION OF THE INVENTION

Various preferred features and embodiments of the present invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

Blood-Brain Barrier (BBB)

The blood-brain barrier (BBB) is a selective barrier that separates circulating blood from the brain. The BBB is comprised of a monolayer of endothelial cells bonded by tight junction proteins that form the small cerebral blood vessel lumen. In addition, astrocytes (in particular, projections from those cells termed astrocytic feet) and pericytes contribute to the structure and function of the BBB.

The BBB governs entry of all peripherally circulating factors such as water diffusion, some gases and lipid-soluble molecules, and selective transport of other substances, such as glucose, amino acids, and micronutrients that are crucial to neuronal function. Conversely, the BBB protects the brain from the passage of toxic substances that may place the central nervous system (CNS) at risk.

The term “impaired blood-brain barrier (BBB)” refers to a BBB that is not functioning correctly as a selective barrier between circulation and the brain. As used herein, the term “impaired blood-brain barrier (BBB)” may be equated with “dysfunctional blood-brain barrier (BBB)”.

One example, is the case where certain larger proteins that are more abundant in circulation begin to penetrate the BBB (“leak”) and infiltrate the cerebrospinal fluid (CSF) would be a case of impaired BBB.

An impaired BBB may occur, for example, in a subject having a higher than normal CSF-to-serum albumin ratio, for example a CSF-to-serum albumin ratio greater than or equal to 5, 6, 7, 8, or 9, preferably greater than or equal to 9.

Cognitive Impairment

A number of studies have observed BBB dysfunction with all forms of dementia, including Alzheimer's disease. For example, post-mortem analyses have demonstrated BBB damage in Alzheimer's disease patients. In addition, neuroimaging studies have shown the accumulation of iron and microbleeds in Alzheimer's disease patients, which suggests subtle haemorrhage or rupture of small vessels in the brain at some point across the lifespan. Further studies have shown that cerebrospinal fluid (CSF)-to-serum ratios of blood-derived albumin are higher in all dementia patients (including those suffering from Alzheimer's disease) when compared against age-matched controls. Indeed, this measure of BBB function associates with accelerated Alzheimer's disease progression independent of age and other Alzheimer's disease risk factors.

BBB dysfunction therefore appears to be a significant risk factor for the development of cognitive impairments, such as Alzheimer's disease, and their progression.

The term “cognition” refers to the set of mental thinking abilities and domains of attention and processing speed, short and long term memory, working memory, executive functions of planning and flexibility, decision making, judgment and evaluation, reasoning and “computation”, problem solving, comprehension and language. “Cognitive impairment” refers to a deterioration in one or more these domains of cognition.

Levels of and improvements in cognition can be readily assessed by the skilled person using any of a number of validated neuropsychological tests standardized to assess, for example, speed of information processing, executive function and memory.

Suitable example tests include Mini Mental State Examination (MMSE), Clinical Dementia Rating (CDR), Cambridge Neuropsychological Test Automated Battery (CANTAB), Alzheimer's Disease Assessment Scale-cognitive test (ADAScog), Wisconsin Card Sorting Test, Verbal and Figural Fluency Test and Trail Making Test.

In addition, medical imaging of the brain provides an assessment of brain function. Examples of medical imaging techniques used for assessment of brain function include electroencephalography (EEG), magnetoencephalography (MEG), Positron Emission Tomography (PET), Single Photon Emission Computed Tomography (SPECT), Magnetic Resonance Imaging (MRI), functional Magnetic Resonance Imaging (fMRI), computerised tomography and long-term potentiation. Dynamic gadolinium enhanced MRI can also be used to assess blood brain barrier (BBB) function.

EEG, a measure of electrical activity of the brain, is accomplished by placing electrodes on the scalp at various landmarks and recording greatly amplified brain signals. MEG is similar to EEG in that it measures the magnetic fields that are linked to electrical fields. MEG is used to measure spontaneous brain activity, including synchronous waves in the nervous system.

PET provides a measure of oxygen utilisation and glucose metabolism. In this technique, a radioactive positron-emitting tracer is administered, and tracer uptake by the brain is correlated with brain activity. These tracers emit gamma rays which are detected by sensors surrounding the head, resulting in a 3D map of brain activation. As soon as the tracer is taken up by the brain, the detected radioactivity occurs as a function of regional cerebral blood flow. During activation, an increase in cerebral blood flow and neuronal glucose metabolism can be detected within seconds.

Suitable analysis can also be based on neuropsychological testing, general and neurological examinations and individual complaints of cognitive decline (e.g. subjective memory loss).

Cognitive impairment may be, for example, interpreted as a statistically significant difference in performance at any time point in a suitable test.

Alzheimer's Disease (AD)

Alzheimer's disease is caused by atrophy of areas of the brain. Although it is not known what initiates the atrophy, studies have found amyloid plaques, neurofibrillary tangles and acetylcholine imbalances in the brains of Alzheimer's patients. Vascular damage in the brain, which may damage healthy neurons, is also common in Alzheimer's patients.

Alzheimer's disease is a progressive condition that affects multiple brain functions. Early signs of the disease usually include minor memory problems, for example forgetting recent events or the names of places and objects. As the disease progresses, memory problems become more severe and additional symptoms can develop, such as confusion, disorientation, difficulty making decisions, problems with speech and language, and personality changes.

Vascular Dementia

Vascular dementia results from reduced blood flow to the brain, which damages brain cells. The reduced blood flow can occur for a number of reasons, including narrowing of the blood vessels in the brain (subcortical vascular dementia), stroke (single-infarct dementia) and numerous small strokes (multi-infarct dementia). The reduced blood flow may additionally be caused by Alzheimer's disease, a combination referred to as mixed dementia.

Early symptoms of vascular dementia include slowness of thought, difficulty with planning, difficulty with language, problems with attention and concentration, and behavioural changes. The symptoms typically worsen in steps, with intervening stable periods of months or years.

Parkinson's Disease (PD)

Parkinson's disease is a condition in which nerve cells in the substantia nigra become progressively damaged. Nerve cells in this area of the brain produce dopamine, which acts as a messenger between the parts of the brain and nervous system that control body movement. Damage to these nerve cells results in a reduction in the amount of dopamine produced in the brain, which has the effect of reducing function in the part of the brain controlling movement.

Symptoms of the Parkinson's disease include tremors, slow movement, and stiff and inflexible muscles. Parkinson's disease patients may also experience additional symptoms, including depression, constipation, insomnia, anosmia and memory problems.

Age-Related Cognitive Decline

Age-related cognitive decline is the normal, non-pathological reduction in cognitive function that is associated with ageing. Although certain mental functions exhibit little age-related decline (e.g. language, reading and vocabulary skills, some numerical abilities and general knowledge) others decline from middle age (e.g. episodic memory, executive functions, speed of processing and reasoning). The extent to which subjects are affected by age-related cognitive decline varies between individuals.

Age-related cognitive decline usually is not considered severe enough to meet criteria for mild-cognitive impairment. Mild cognitive impairment (MCI) is considered to be objective assessment of cognitive deficit in at least one cognitive domain (age and gender adjusted) that does not impair activities of daily living. In contrast, probable Alzheimer's disease diagnosis requires impairment in at least two cognitive domains and impairment of activities of daily living.

Traumatic Brain Injury (TBI)

Traumatic brain injury is a non-congenital insult to the brain from an external mechanical force, possibly leading to permanent or temporary impairment of cognitive, physical, and psychosocial functions, with an associated diminished or altered state of consciousness.

Biomarkers

Serum Amyloid A (SAA)

Serum amyloid A (SAA) proteins are apolipoproteins that are associated with high-density lipoprotein (HDL) in plasma and are mainly produced by the liver.

In one embodiment, the SAA is human SAA.

A number of isoforms of human SAA are known. In one embodiment, the SAA is SAA1, SAA2 or SAA4, preferably SAA1.

An example amino acid sequence of SAA1 is the sequence deposited under NCBI Accession No. NP_000322.2.

A further example amino acid sequence of SAA1 is:

(SEQ ID NO: 1)
MKLLTGLVFCSLVLGVSSRSFFSFLGEAFDGARDMWRAYSDMREANYIGS
DKYFHARGNYDAAKRGPGGVWAAEAISDARENIQRFFGHGAEDSLADQAA
NEWGRSGKDPNHFRPAGLPEKY

A further example amino acid sequence of SAA1 is:

(SEQ ID NO: 2)
MKLLTGLVFCSLVLGVSSRSFFSFLGEAFDGARDMWRAYSDMREANYIGS
DKYFHARGNYDAAKRGPGGAWAAEVISDARENIQRFFGHGAEDSLADQAA
NEWGRSGKDPNHFRPAGLPEKY

SAA1 may be processed into a mature form, for example by cleavage of a signal peptide. Thus, a further example amino acid sequence of SAA1 is:

(SEQ ID NO: 3)
RSFFSFLGEAFDGARDMWRAYSDMREANYIGSDKYFHARGNYDAAKRGPG
GAWAAEVISDARENIQRFFGHGAEDSLADQAANEWGRSGKDPNHFRPAGL
PEKY

SAA2 has two splice variants. An example amino acid sequence of SAA2 is the sequence deposited under NCBI Accession No. NP_110381.2.

A further example amino acid sequence of SAA2 is:

(SEQ ID NO: 4)
MKLLTGLVFCSLVLSVSSRSFFSFLGEAFDGARDMWRAYSDMREANYI
GSDKYFHARGNYDAAKRGPGGAWAAEVISNARENIQRLTGRGAEDSLA
DQAANKWGRSGRDPNHFRPAGLPEKY

SAA2 may be processed into a mature form, for example by cleavage of a signal peptide. Thus, a further example amino acid sequence of SAA2 is:

(SEQ ID NO: 5)
RSFFSFLGEAFDGARDMWRAYSDMREANYIGSDKYFHARGNYDAAKRG
PGGAWAAEVISNARENIQRLTGRGAEDSLADQAANKWGRSGRDPNHFR
PAGLPEKY

A further example amino acid sequence of SAA2 is the sequence deposited under NCBI Accession No. NP 001120852.1.

A further example amino acid sequence of SAA2 is:

(SEQ ID NO: 6)
MKLLTGLVFCSLVLSVSSRSFFSFLGEAFDGARDMWRAYSDMREANYI
GSDKYFHARGNYDAAKRGPGGAWAAEVISLFSAEL

SAA2 may be processed into a mature form, for example by cleavage of a signal peptide. Thus, a further example amino acid sequence of SAA2 is:

(SEQ ID NO: 7)
RSFFSFLGEAFDGARDMWRAYSDMREANYIGSDKYFHARGNYDAAKRG
PGGAWAAEVISLFSAEL

An example amino acid sequence of SAA4 is the sequence deposited under NCBI Accession No. NP_006503.2.

A further example amino acid sequence of SAA4 is:

(SEQ ID NO: 8)
MRLFTGIVFCSLVMGVTSESWRSFFKEALQGVGDMGRAYWDIMISNHQ
NSNRYLYARGNYDAAQRGPGGVWAAKLISRSRVYLQGLIDCYLFGNSS
TVLEDSKSNEKAEEWGRSGKDPDRFRPDGLPKKY

SAA4 may be processed into a mature form, for example by cleavage of a signal peptide. Thus, a further example amino acid sequence of SAA4 is:

(SEQ ID NO: 9)
ESWRSFFKEALQGVGDMGRAYWDIMISNHQNSNRYLYARGNYDAAQRG
PGGVWAAKLISRSRVYLQGLIDCYLFGNSSTVLEDSKSNEKAEEWGRS
GKDPDRFRPDGLPKKY

Macrophage-Derived Chemokine (MDC)

The macrophage-derived chemokine (MDC) protein is secreted by dendritic cells and macrophages. MDC interacts with cell surface chemokine receptors, such as CCR4, to elicit effects on target cells and it may be involved in the trafficking of activated/effector T lymphocytes to inflammatory sites.

MDC is also known as C-C motif chemokine 22 (CCL22).

In one embodiment, the MDC is human MDC.

An example amino acid sequence of MDC is the sequence deposited under NCBI Accession No. NP_002981.2.

A further example amino acid sequence of MDC is:

(SEQ ID NO: 10)
MDRLQTALLVVLVLLAVALQATEAGPYGANMEDSVCCRDYVRYRLPLR
VVKHFYWTSDSCPRPGVVLLTFRDKEICADPRVPWVKMILNKLSQ

MDC may be processed into a mature form, for example by cleavage of a signal peptide. Thus, a further example amino acid sequence of MDC is:

(SEQ ID NO: 11)
GPYGANMEDSVCCRDYVRYRLPLRVVKHFYWTSDSCPRPGVVLLTFRD
KEICADPRVPWVKMILNKLSQ

Soluble Inter-Cellular Adhesion Molecule-1 (sICAM-1)

Soluble inter-cellular adhesion molecule-1 (sICAM-1) is a member of the soluble cell adhesion molecule (sCAM) class of cell surface binding proteins. In particular, sICAM-1 is a soluble form of the iCAM-1 cell adhesion molecule.

In one embodiment, the sICAM-1 is human sICAM-1. An example of human sICAM-1 is:

(SEQ. ID No. 12)
ESVTVTRDLEGTYLCRARSTQGEVTREPPGMRLSSSLW

Vascular Endothelial Growth Factor (VEGF)

Vascular endothelial growth factor (VEGF) is a signalling protein, which stimulates vasculogenesis and angiogenesis.

In one embodiment, the VEGF is human VEGF.

In one embodiment, the VEGF is VEGF-A, VEGF-B, VEGF-C, VEGF-D or placenta growth factor (PGF), preferably the VEGF is VEGF-A.

An example amino acid sequence of VEGF is the sequence deposited under NCBI Accession No. NP_001165094.1.

A further example amino acid sequence of VEGF is:

(SEQ ID NO: 13)
MNFLLSWVHWSLALLLYLHHAKWSQAAPMAEGGGQNHHEVVKFMDVYQ
RSYCHPIETLVDIFQEYPDEIEYIFKPSCVPLMRCGGCCNDEGLECVP
TEESNITMQIMRIKPHQGQHIGEMSFLQHNKCECRPKKDRARQEKKSV
RGKGKGQKRKRKKSRYKSWSVYVGARCCLMPWSLPGPHPCGPCSERRK
HLFVQDPQTCKCSCKNTDSRCKARQLELNERTCRCDKPRR

VEGF may be processed into a mature form, for example by cleavage of a signal peptide. Thus, a further example amino acid sequence of VEGF is:

(SEQ ID NO: 14)
APMAEGGGQNHHEVVKFMDVYQRSYCHPIETLVDIFQEYPDEIEYIFK
PSCVPLMRCGGCCNDEGLECVPTEESNITMQIMRIKPHQGQHIGEMSF
LQHNKCECRPKKDRARQEKKSVRGKGKGQKRKRKKSRYKSWSVYVGAR
CCLMPWSLPGPHPCGPCSERRKHLFVQDPQTCKCSCKNTDSRCKARQL
ELNERTCRCDKPRR

Further examples of amino acid sequence VEGF-A isoform are:

(SEQ ID NO: 15)
MNFLLSWVHWSLALLLYLHHAKWSQAAPMAEGGGQNHHEVVKFMDVYQ
RSYCHPIETLVDIFQEYPDEIEYIFKPSCVPLMRCGGCCNDEGLECVP
TEESNITMQIMRIKPHQGQHIGEMSFLQHNKCECRPKKDRARQEKKSV
RGKGKGQKRKRKKSRYKSWSVYVGARCCLMPWSLPGPHPCGPCSERRK
HLFVQDPQTCKCSCKNTDSRCKARQLELNERTCRCDKPRR
(SEQ ID NO: 16)
MNFLLSWVHWSLALLLYLHHAKWSQAAPMAEGGGQNHHEVVKFMDVYQ
RSYCHPIETLVDIFQEYPDEIEYIFKPSCVPLMRCGGCCNDEGLECVP
TEESNITMQIMRIKPHQGQHIGEMSFLQHNKCECRPKKDRARQEKKSV
RGKGKGQKRKRKKSRYKSWSVPCGPCSERRKHLFVQDPQTCKCSCKNT
DSRCKARQLELNERTCRCDKPRR
(SEQ ID NO: 17)
MNFLLSWVHWSLALLLYLHHAKWSQAAPMAEGGGQNHHEVVKFMDVYQ
RSYCHPIETLVDIFQEYPDEIEYIFKPSCVPLMRCGGCCNDEGLECVP
TEESNITMQIMRIKPHQGQHIGEMSFLQHNKCECRPKKDRARQEKKSV
RGKGKGQKRKRKKSRPCGPCSERRKHLFVQDPQTCKCSCKNTDSRCKA
RQLELNERTCRCDKPRR
(SEQ ID NO: 18)
MNFLLSWVHWSLALLLYLHHAKWSQAAPMAEGGGQNHHEVVKFMDVYQ
RSYCHPIETLVDIFQEYPDEIEYIFKPSCVPLMRCGGCCNDEGLECVP
TEESNITMQIMRIKPHQGQHIGEMSFLQHNKCECRPKKDRARQENPCG
PCSERRKHLFVQDPQTCKCSCKNTDSRCKARQLELNERTCRCDKPRR
(SEQ ID NO: 19)
MNFLLSWVHWSLALLLYLHHAKWSQAAPMAEGGGQNHHEVVKFMDVYQ
RSYCHPIETLVDIFQEYPDEIEYIFKPSCVPLMRCGGCCNDEGLECVP
TEESNITMQIMRIKPHQGQHIGEMSFLQHNKCECRPKKDRARQENPCG
PCSERRKHLFVQDPQTCKCSCKNTDSRCKM
(SEQ ID NO: 20)
MNFLLSWVHWSLALLLYLHHAKWSQAAPMAEGGGQNHHEVVKFMDVYQ
RSYCHPIETLVDIFQEYPDEIEYIFKPSCVPLMRCGGCCNDEGLECVP
TEESNITMQIMRIKPHQGQHIGEMSFLQHNKCECRPKKDRARQEKKSV
RGKGKGQKRKRKKSRYKSWSVCDKPRR
(SEQ ID NO: 21)
MNFLLSWVHWSLALLLYLHHAKWSQAAPMAEGGGQNHHEVVKFMDVYQ
RSYCHPIETLVDIFQEYPDEIEYIFKPSCVPLMRCGGCCNDEGLECVP
TEESNITMQIMRIKPHQGQHIGEMSFLQHNKCECRPKKDRARQENPCG
PCSERRKHLFVQDPQTCKCSCKNTDSRCKARQLELNERTCRSLTRKD
(SEQ ID NO: 22)
MNFLLSWVHWSLALLLYLHHAKWSQAAPMAEGGGQNHHEVVKFMDVYQ
RSYCHPIETLVDIFQEYPDEIEYIFKPSCVPLMRCGGCCNDEGLECVP
TEESNITMQIMRIKPHQGQHIGEMSFLQHNKCECRPKKDRARQEKCDK
PRR
(SEQ ID NO: 23)
MNFLLSWVHWSLALLLYLHHAKWSQAAPMAEGGGQNHHEVVKFMDVYQ
RSYCHPIETLVDIFQEYPDEIEYIFKPSCVPLMRCGGCCNDEGLECVP
TEESNITMQIMRIKPHQGQHIGEMSFLQHNKCECRCDKPRR
(SEQ ID NO: 24)
MTDRQTDTAPSPSYHLLPGRRRTVDAAASRGQGPEPAPGGGVEGVGAR
GVALKLFVQLLGCSRFGGAVVRAGEAEPSGAARSASSGREEPQPEEGE
EEEEKEEERGPQWRLGARKPGSWTGEAAVCADSAPAARAPQALARASG
RGGRVARRGAEESGPPHSPSRRGSASRAGPGRASETMNFLLSWVHWSL
ALLLYLHHAKWSQAAPMAEGGGQNHHEVVKFMDVYQRSYCHPIETLVD
IFQEYPDEIEYIFKPSCVPLMRCGGCCNDEGLECVPTEESNITMQIMR
IKPHQGQHIGEMSFLQHNKCECRPKKDRARQENPCGPCSERRKHLFVQ
DPQTCKCSCKNTDSRCKARQLELNERTCRCDKPRR
(SEQ ID NO: 25)
MTDRQTDTAPSPSYHLLPGRRRTVDAAASRGQGPEPAPGGGVEGVGAR
GVALKLFVQLLGCSRFGGAVVRAGEAEPSGAARSASSGREEPQPEEGE
EEEEKEEERGPQWRLGARKPGSWTGEAAVCADSAPAARAPQALARASG
RGGRVARRGAEESGPPHSPSRRGSASRAGPGRASETMNFLLSWVHWSL
ALLLYLHHAKWSQAAPMAEGGGQNHHEVVKFMDVYQRSYCHPIETLVD
IFQEYPDEIEYIFKPSCVPLMRCGGCCNDEGLECVPTEESNITMQIMR
IKPHQGQHIGEMSFLQHNKCECRPKKDRARQEKCDKPRR
(SEQ ID NO: 26)
MTDRQTDTAPSPSYHLLPGRRRTVDAAASRGQGPEPAPGGGVEGVGAR
GVALKLFVQLLGCSRFGGAVVRAGEAEPSGAARSASSGREEPQPEEGE
EEEEKEEERGPQWRLGARKPGSWTGEAAVCADSAPAARAPQALARASG
RGGRVARRGAEESGPPHSPSRRGSASRAGPGRASETMNFLLSWVHWSL
ALLLYLHHAKWSQAAPMAEGGGQNHHEVVKFMDVYQRSYCHPIETLVD
IFQEYPDEIEYIFKPSCVPLMRCGGCCNDEGLECVPTEESNITMQIMR
IKPHQGQHIGEMSFLQHNKCECRPKKDRARQEKKSVRGKGKGQKRKRK
KSRYKSWSVPCGPCSERRKHLFVQDPQTCKCSCKNTDSRCKARQLELN
ERTCRCDKPRR
(SEQ ID NO: 27)
MTDRQTDTAPSPSYHLLPGRRRTVDAAASRGQGPEPAPGGGVEGVGAR
GVALKLFVQLLGCSRFGGAVVRAGEAEPSGAARSASSGREEPQPEEGE
EEEEKEEERGPQWRLGARKPGSWTGEAAVCADSAPAARAPQALARASG
RGGRVARRGAEESGPPHSPSRRGSASRAGPGRASETMNFLLSWVHWSL
ALLLYLHHAKWSQAAPMAEGGGQNHHEVVKFMDVYQRSYCHPIETLVD
IFQEYPDEIEYIFKPSCVPLMRCGGCCNDEGLECVPTEESNITMQIMR
IKPHQGQHIGEMSFLQHNKCECRPKKDRARQEKKSVRGKGKGQKRKRK
KSRYKSWSVYVGARCCLMPWSLPGPHPCGPCSERRKHLFVQDPQTCKC
SCKNTDSRCKARQLELNERTCRCDKPRR
(SEQ ID NO: 28)
MTDRQTDTAPSPSYHLLPGRRRTVDAAASRGQGPEPAPGGGVEGVGAR
GVALKLFVQLLGCSRFGGAVVRAGEAEPSGAARSASSGREEPQPEEGE
EEEEKEEERGPQWRLGARKPGSWTGEAAVCADSAPAARAPQALARASG
RGGRVARRGAEESGPPHSPSRRGSASRAGPGRASETMNFLLSWVHWSL
ALLLYLHHAKWSQAAPMAEGGGQNHHEVVKFMDVYQRSYCHPIETLVD
IFQEYPDEIEYIFKPSCVPLMRCGGCCNDEGLECVPTEESNITMQIMR
IKPHQGQHIGEMSFLQHNKCECRPKKDRARQENPCGPCSERRKHLFVQ
DPQTCKCSCKNTDSRCKARQLELNERTCRSLTRKD
(SEQ ID NO: 29)
MTDRQTDTAPSPSYHLLPGRRRTVDAAASRGQGPEPAPGGGVEGVGAR
GVALKLFVQLLGCSRFGGAVVRAGEAEPSGAARSASSGREEPQPEEGE
EEEEKEEERGPQWRLGARKPGSWTGEAAVCADSAPAARAPQALARASG
RGGRVARRGAEESGPPHSPSRRGSASRAGPGRASETMNFLLSWVHWSL
ALLLYLHHAKWSQAAPMAEGGGQNHHEVVKFMDVYQRSYCHPIETLVD
IFQEYPDEIEYIFKPSCVPLMRCGGCCNDEGLECVPTEESNITMQIMR
IKPHQGQHIGEMSFLQHNKCECRPKKDRARQEKKSVRGKGKGQKRKRK
KSRPCGPCSERRKHLFVQDPQTCKCSCKNTDSRCKARQLELNERTCRC
DKPRR
(SEQ ID NO: 30)
MTDRQTDTAPSPSYHLLPGRRRTVDAAASRGQGPEPAPGGGVEGVGAR
GVALKLFVQLLGCSRFGGAVVRAGEAEPSGAARSASSGREEPQPEEGE
EEEEKEEERGPQWRLGARKPGSWTGEAAVCADSAPAARAPQALARASG
RGGRVARRGAEESGPPHSPSRRGSASRAGPGRASETMNFLLSWVHWSL
ALLLYLHHAKWSQAAPMAEGGGQNHHEVVKFMDVYQRSYCHPIETLVD
IFQEYPDEIEYIFKPSCVPLMRCGGCCNDEGLECVPTEESNITMQIMR
IKPHQGQHIGEMSFLQHNKCECRPKKDRARQENPCGPCSERRKHLFVQ
DPQTCKCSCKNTDSRCKM
(SEQ ID NO: 31)
MTDRQTDTAPSPSYHLLPGRRRTVDAAASRGQGPEPAPGGGVEGVGAR
GVALKLFVQLLGCSRFGGAVVRAGEAEPSGAARSASSGREEPQPEEGE
EEEEKEEERGPQWRLGARKPGSWTGEAAVCADSAPAARAPQALARASG
RGGRVARRGAEESGPPHSPSRRGSASRAGPGRASETMNFLLSWVHWSL
ALLLYLHHAKWSQAAPMAEGGGQNHHEVVKFMDVYQRSYCHPIETLVD
IFQEYPDEIEYIFKPSCVPLMRCGGCCNDEGLECVPTEESNITMQIMR
IKPHQGQHIGEMSFLQHNKCECRCDKPRR
Further examples of VEGF-B isoform are:
(SEQ ID NO: 32)
MSPLLRRLLLAALLQLAPAQAPVSQPDAPGHQRKVVSWIDVYTRATCQ
PREVVVPLTVELMGTVAKQLVPSCVTVQRCGGCCPDDGLECVPTGQHQ
VRMQILMIRYPSSQLGEMSLEEHSQCECRPKKKDSAVKPDRAATPHHR
PQPRSVPGWDSAPGAPSPADITHPTPAPGPSAHAAPSTTSALTPGPAA
AAADAAASSVAKGGA
(SEQ ID NO: 33)
MSPLLRRLLLAALLQLAPAQAPVSQPDAPGHQRKVVSWIDVYTRATCQ
PREVVVPLTVELMGTVAKQLVPSCVTVQRCGGCCPDDGLECVPTGQHQ
VRMQILMIRYPSSQLGEMSLEEHSQCECRPKKKDSAVKPDSPRPLCPR
CTQHHQRPDPRTCRCRCRRRSFLRCQGRGLELNPDTCRCRKLRR
(SEQ ID NO: 34)
MHLLGFFSVACSLLAAALLPGPREAPAAAAAFESGLDLSDAEPDAGEA
TAYASKDLEEQLRSVSSVDELMTVLYPEYWKMYKCQLRKGGWQHNREQ
ANLNSRTEETIKFAAAHYNTEILKSIDNEWRKTQCMPREVCIDVGKEF
GVATNTFFKPPCVSVYRCGGCCNSEGLQCMNTSTSYLSKTLFEITVPL
SQGPKPVTISFANHTSCRCMSKLDVYRQVHSIIRRSLPATLPQCQAAN
KTCPTNYMWNNHICRCLAQEDFMFSSDAGDDSTDGFHDICGPNKELDE
ETCQCVCRAGLRPASCGPHKELDRNSCQCVCKNKLFPSQCGANREFDE
NTCQCVCKRTCPRNQPLNPGKCACECTESPQKCLLKGKKFHHQTCSCY
RRPCTNRQKACEPGFSYSEEVCRCVPSYWKRPQMS

An example of VEGF-C isoform is:

An example of VEGF-D isoform is:

(SEQ ID NO: 35)
MYREWVVVNVFMMLYVQLVQGSSNEHGPVKRSSQSTLERSEQQIRAAS
SLEELLRITHSEDWKLWRCRLRLKSFTSMDSRSASHRSTRFAATFYDI
ETLKVIDEEWQRTQCSPRETCVEVASELGKSTNTFFKPPCVNVFRCGG
CCNEESLICMNTSTSYISKQLFEISVPLTSVPELVPVKVANHTGCKCL
PTAPRHPYSIIRRSIQIPEEDRCSHSKKLCPIDMLWDSNKCKCVLQEE
NPLAGTEDHSHLQEPALCGPHMMFDEDRCECVCKTPCPKDLIQHPKNC
SCFECKESLETCCQKHKLFHPDTCSCEDRCPFHTRPCASGKTACAKHC
RFPKEKRAAQGPHSRKNP

Interleukin 8 (IL-8)

Interleukin 8 (IL-8) is a chemokine that is produced by macrophages, epithelial cells, airway smooth muscle cells and endothelial cells. IL-8 binds to a number of cell-surface receptors, including CXCR1 and CXCR2, and is an important mediator of the innate immune response.

IL-8 is also known as chemokine (C-X-C motif) ligand 8 (CXCL8).

In one embodiment, the IL-8 is human IL-8.

An example amino acid sequence of IL-8 is the sequence deposited under NCBI Accession No. NP_000575.1.

A further example amino acid sequence of IL-8 is:

(SEQ ID NO: 36)
MTSKLAVALLAAFLISAALCEGAVLPRSAKELRCQCIKTYSKPFHPKF
IKELRVIESGPHCANTEIIVKLSDGRELCLDPKENWVQRVVEKFLKRA
ENS

IL-8 may be processed into a mature form, for example by cleavage of a signal peptide. Thus, a further example amino acid sequence of IL-8 is:

(SEQ ID NO: 37)
AVLPRSAKELRCQCIKTYSKPFHPKFIKELRVIESGPHCANTEIIVKL
SDGRELCLDPKENWVQRVVEKFLKRAENS

Determining Biomarker Levels

The level of the individual biomarker species in the sample may be measured or determined by any suitable method known in the art. For example, mass spectrometry (MS), antibody-based detection methods (e.g. enzyme-linked immunosorbent assay, ELISA), non-antibody protein scaffold-based methods (e.g. fibronectin scaffolds), radioimmunoassays (RIA) or aptamer-based methods may be used. Other spectroscopic methods, chromatographic methods, labelling techniques or quantitative chemical methods may also be used.

In one embodiment, the level of the one or more biomarkers may be determined via binding to one or more antibodies that are specific to the one or more biomarkers. Suitable antibodies are known or may be generated using known techniques.

Suitable methods for detecting antibody levels include, but are not limited to, immunoassays, such as enzyme-linked immunosorbent assays (ELISAs), radioimmunoassays, Western blotting and immunoprecipitation.

Preferably, the level of the one or more biomarkers is determined using a sandwich immunoassay.

The antibody may be, for example, a monoclonal antibody, polyclonal antibody, multispecific antibody (e.g. bispecific antibody) or fragment thereof provided that it specifically binds to the biomarker being detected. Antibodies may be obtained by standard techniques comprising immunising an animal with a target antigen and isolating the antibody from serum. Monoclonal antibodies may be made by the hybridoma method first described by Kohler et al. (Kohler et al. (1975) Nature 256: 495) or may be made by recombinant DNA methods (e.g. disclosed in U.S. Pat. No. 4,816,567). Monoclonal antibodies may also be isolated from phage antibody libraries using the techniques described in Clackson et al. (Clackson et al. (1991) Nature 352: 624-628) and Marks et al. (Marks et al. (1991) J. Mol. Biol. 222: 581-597), for example. The antibody may also be a chimeric or humanised antibody.

In one embodiment, the level of the one or more biomarkers may be determined by staining the sample with a reagent that labels one or more of the biomarkers. “Staining” is typically a histological method, which renders the biomarker detectable, for example by microscopic techniques, such as those using visible or fluorescent light.

In one embodiment, the biomarker is detected in the sample by immunohistochemistry (IHC). In IHC, the biomarker may be detected by an antibody that binds specifically to one or more of the biomarkers.

Two general methods of antibody-based detection (including for IHC-based methods) are available: direct and indirect assays. According to the first assay, binding of antibody to the target antigen is determined directly. This direct assay uses a labelled reagent, such as a fluorescent tag or an enzyme-labelled primary antibody, which can be visualised without further antibody interaction.

In a typical indirect assay, unconjugated primary antibody binds to the antigen and then a labelled secondary antibody binds to the primary antibody. Where the secondary antibody is conjugated to an enzymatic label, a chromogenic or fluorogenic substrate is added to provide visualisation of the antigen. Signal amplification occurs because several secondary antibodies may react with different epitopes on the primary antibody.

The primary and/or secondary antibody used may be labelled with a detectable moiety. Numerous labels are available, including radioisotopes, colloidal gold particles, fluorescent labels and various enzyme-substrate labels. Fluorescent labels include, but are not limited to, rare earth chelates (europium chelates), Texas Red, rhodamine, fluorescein, dansyl, Lissamine, umbelliferone, phycocrytherin and phycocyanin, and/or derivatives of any one or more of the above. The fluorescent labels can be conjugated to the antibody using known techniques.

Various enzyme-substrate labels are available (e.g. disclosed in U.S. Pat. No. 4,275,149). The enzyme generally catalyses a chemical alteration of the chromogenic substrate that can be detected microscopically, for example under visible light. For example, the enzyme may catalyse a colour change in a substrate, or may alter the fluorescence or chemiluminescence of the substrate. Examples of enzymatic labels include luciferases (e.g. firefly luciferase and bacterial luciferase; e.g. disclosed in U.S. Pat. No. 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g. glucose oxidase, galactose oxidase and glucose-6-phosphate dehydrogenase), heterocyclic oxidases (e.g. uricase and xanthine oxidase), lactoperoxidase, microperoxidase, and the like. Techniques for conjugating enzymes to antibodies are well known.

Typically IHC methods may comprise a step of detecting stained regions within an image. Pixels in the image corresponding to staining associated with the biomarker may be identified by colour transformation methods, for example as disclosed in U.S. Pat. Nos. 6,553,135 and 6,404,916. In such methods, stained objects of interest may be identified by recognising the distinctive colour associated with the stain. The method may comprise transforming pixels of the image to a different colour space and applying a threshold value to suppress background staining.

For example, a ratio of two of the RGB signal values may be formed to provide a means for discriminating colour information. A particular stain may be discriminated from background by the presence of a minimum value for a particular signal ratio. For example, pixels corresponding to a predominantly red stain may be identified by a ratio of red divided by blue (RIB) which is greater than a minimum value.

Kong et al. (Kong et al. (2013) Am. J. Clin. Nutr. 98: 1385-94) describes the use of the avidin-biotin-peroxidase method and two independent investigators counting the number of positively stained cells.

Detection using aptamers may comprise the following steps:
aptamers that specifically recognise the biomarker may be synthesised using standard nucleic acid synthesis techniques or selected from a large random sequence pool, for example using the Systematic Evolution of Ligands by Exponential Enrichment (SELEX) technique;
aptamers are mixed with the samples so that aptamer-protein complexes are formed;
non-specific complexes are separated;
bound aptamers are removed from their target proteins;
aptamers are collected and measured, for example using microarrays or mass spectrometry techniques.

Aptamers can be single stranded DNA or RNA sequences that fold into a unique 3D structure having a combination of stems, loops, quadruplexes, pseudoknots, bulges or hairpins. The molecular recognition of aptamers results from intermolecular interactions, such as the stacking of aromatic rings, electrostatic and van der Waals interactions, or hydrogen bonding with a target compound. In addition, the specific interaction between an aptamer and its target is complemented through an induced fit mechanism, which requires the aptamer to adopt a unique folded structure to its target. Aptamers can be modified to be linked with labelling molecules such as dyes or immobilised on the surface of beads or substrates for different applications.

Samples

The invention comprises a step of determining the level of one or more biomarkers in one or more samples obtained from a subject.

Preferably, the sample is cerebrospinal fluid (CSF) sample or a sample derived from blood.

The sample derived from blood may contain a blood fraction or may be whole blood. Preferably, the sample derived from blood is a plasma or serum sample, most preferably a serum sample.

Techniques for collecting samples from a subject are well known in the art.

Subject

The subjects disclosed herein are preferably mammals, particularly preferably humans. Both human and veterinary applications are within the scope of the invention.

The subject may be, for example, an ageing human subject, such as a human over the age of 30, 35, 40, 45, 50, 55, 60, 65, 70, 75, 80, 85, 90, 95 or 100 years old. Preferably, the subject is a human over the age of 55 years old. For veterinary applications, the age of the animal would be scaled from the human situation using the average lifespan for calibration.

Method of Treatment

It is to be appreciated that all references herein to treatment include curative, palliative and prophylactic treatment; although in the context of the invention references to preventing are more commonly associated with prophylactic treatment. Treatment may also include arresting progression in the severity of a disease.

Dietary Intervention

The term “dietary intervention” refers to an external factor applied to a subject which causes a change in the subject's diet.

In one embodiment, the dietary intervention is a diet supplemented with vitamins and/or minerals, preferably vitamin B.

In another embodiment, the dietary intervention is a diet supplemented with omega-3 fatty acids.

In one embodiment, the dietary intervention comprises increasing vitamin B intake by the subject, preferably by administering a vitamin B supplement.

In another embodiment, the dietary intervention comprises increasing omega-3 fatty acid intake by the subject, preferably by administering an omega-3 fatty acid supplement.

The vitamin B may be, for example, vitamin B12, vitamin B6 and/or folic acid.

The omega-3 fatty acid may be, for example, eicosapentaenoic acid (EPA) or docosahexaenoic acid (DHA), preferably EPA.

The diet may be one which is adjusted to the starting body weight of the subject.

The dietary intervention may comprise administration of at least one diet product. The diet product may be a meal replacement product or a supplement product. The diet product may include food products, drinks, pet food products, food supplements, nutraceuticals, food additives or nutritional formulae.

### EXAMPLES

### Example 1

Materials and Methods

Subject Population

120 community-dwelling adults aged 55 years or older (48 of them having no cognitive impairment, 72 with cognitive impairment (mild cognitive impairment (MCI), n=63; and mild dementia, n=9) were enrolled in this study. The clinical evaluation included a neurological and general examination and extensive neuropsychological evaluation. Subjects with neurological or psychiatric diseases or with a severe or unstable medical illness were excluded. Along with the clinical examination, the Hospital Anxiety and Depression (HAD) scale was administered (Zigmond & Snaith, Acta Psychiatr. Scand. 1983, 67 (6), 361-370).

The study participants with MCI and the participants with mild dementia have been recruited among outpatients with cognitive impairment referred to the Memory Clinics, Departments of Psychiatry, and the Leenaards Memory Center, Department of Clinical Neurosciences, University Hospitals of Lausanne for investigation of their cognitive complaints. The diagnosis of MCI or of mild dementia was based on neuropsychological and clinical evaluation, and made by a consensus conference of psychiatrists and/or neurologists, and neuropsychologists prior to the inclusion in the study. For instance, MCI criteria required memory impairment (<1.5 SD below the age, gender and education adjusted mean on the Buschke Double Memory Test verbal memory score) (Buschke, Sliwinski, Kuslansky, & Lipton, Neurology 1997, 48 (4), 989-997), and/or impairment in another cognitive domain such as executive tasks, and a Clinical Dementia Rating (CDR) (Morris, Neurology 1993, 43 (11), 2412-2414) equal to 0.5.

Probable Alzheimer's dementia was defined according to the clinical diagnostic criteria from the National Institute on Aging and Alzheimer's Association and DSM-IV criteria for dementia of the Alzheimer type (American-Psychiatric-Association). Participants in this group have a CDR of 1.0. The participants without cognitive impairment (n=48) had no history or evidence of cognitive decline, and a CDR score of 0. They are community-dwelling volunteers recruited by advertisement or among the spouses of memory clinic patients.

Neuropsychological and Functional Assessments

The neuropsychological assessment includes measures of memory and other major cognitive domains such as language, attention and executive functioning. This assessment consists of the Mini Mental State Examination (Folstein M F et al. 1975, J. Psychiatr. Res 12, 189-198), the Buschke Double Memory Test (Buschke H et al. 1997, Neurology, 48, 989-997) the digit span forward and backward (Wisdom N M et al. 2012, Arch Clin Neuropsychology 27, 389-397), the Stroop Test (Stroop J R 1935, J. of Expt. Psychology 18, 643-662), the letter fluency task (Cardebat D et al. 1990, Acta Neurol Belg 90, 207-217), and the Trail Making Tests A and B (Reitan R M 1955, J. Consult Psycho) 19, 393-394). The functional assessment includes the ADL and instrumental ADL (IADL) (Lawton M P et al. 1969, Gerontologist 9, 179-186). as well as the CDR (Morris J C 1993, Neurology, 43, 2412-2414). The neuropsychological test battery, ADL and IADL, and the CDR were used to verify inclusion and exclusion criteria.

Additional Assessment

The brief clinical form of the Neuropsychiatric Inventory (Kaufer, D. I. et al. (2000) J. Neuropsychiatry Clin. Neurosci. 12: 233-9) was administrated to assess neuropsychiatric symptoms in all participants. The Cumulative illness rating scale-geriatrics (Miller M D et al. 1992, Psychiatry Res 41, 237-248) was used to measure the participants individual chronic medical illness burden.

Cerebrospinal Fluid (CSF) and Blood Collection and Handling

Venous and lumbar punctures were performed between 8:30 and 9:30 am in the Memory centres after an overnight fast. Blood was drawn into EDTA containing vacutainers (Sarstedt, Germany) and spun down to permit aliquots of supernatant (plasma and serum) for the analysis. Lumbar puncture and spinal fluid collection was performed on subjects in sitting or lying position with a 22G “atraumatical” spinal needle to capture 10-12 mL of CSF into polypropylene tubes. CSF cell count and protein quantification were performed in 2-3 mL and the remaining CSF was centrifuged, aliquoted, snap-frozen and stored at −80° C. until assay.

Neuroinflammatory Biomarker Analysis

A “sandwich” immunoassay (Meso Scale Discovery (MSD), Rockville, Md., USA) quantified 37 analytes (IFN-gamma, IL-1B, IL-2, IL-4, IL-6, IL-8, IL-10, IL-13, TNFa, IL-1a, IL-5, IL-7, IL-12123p40, IL-15, IL-16, IL-17A, TNF-β, VEGFA, Eotaxin, MIP-1B, Eotaxin-3, TARC, IP-10, MIP-1a, MCP-1, MDC, MCP-4, VEGF-C, VEGF-D, Tie-2, Flt-1, PIGF, bFGF, SAA, CRP, VCAM-1, ICAM-1) in CSF and serum.

Samples were measured following the manufacturer's instructions. Briefly, the 96-well plates pre-coated with capture antibodies were blocked with 5% MSD Blocker A Solution. Calibrator dilutions were prepared and samples were diluted as recommended for each kit with MSD Diluents. Samples and calibrators were then added to the plates and incubated at room temperature with shaking for 2 h. Plates were washed three times with a home-prepared solution of 10× phosphate-buffered saline (PBS), pH 7.4 (Corning, Manassas, Va., USA)-Tween 20 (Fisher Scientific, Pittsburgh, Pa., USA). Detection antibodies were mixed with MSD Diluents as indicated in the protocols of each kit and incubated at room temperature with shaking for 1-2 h. Plates were washed three times with the PBS-Tween 20 solution. MSD Read buffer was added and plates were read on an MSD instrument (SECTOR Imager 6000 reader). Data were generated and interpolated using MSD Discovery Workbench software.

APOE Genotyping

Leukocyte genomic DNA was isolated from 9 mL EDTA blood with the Qiagen blood isolation kit (Qiagen, Hilden, Germany) and the APOE genotype was determined.

Ethical Approvals for Human Research

The study was approved by the CHUV (Centre Hospitalier Universitaire Vaudois) Lausanne hospital ethics committee and the Canton of Vaud, Switzerland, Commission Cantonale d'éthique de la recherche sur l'étre humain (CER-VD). Written informed consent was obtained from all study participants.

Statistical Analysis—Pre-Analytical Quality Control of Biomarker Data

Biomarker data was quality-controlled prior to hypothesis testing by first excluding those with more than 5% missing data. The remaining missing data (<5%) was imputed by randomly drawing a measure between the observed range of biomarker values. Biomarker data was then log-transformed to approach a Gaussian distribution, and standardised prior to final hypothesis testing.

Statistical Analysis—Reference Model

The association of BBB impairment with demographic variables (age, gender) and candidate Alzheimer's disease biological parameters (presence of ApoEe4 allele, CDR, education, CSF abeta₁₋₄₂, phospho-tau181 and total tau (t-tau)) was analysed using logistic regression models. The performance of the obtained classifier was assessed by measuring (i) its area under the Receiver Operating Characteristic (ROC) curve and its 95% confidence interval (using a bootstrap approach with 1000 iterations) and (ii) its accuracy (cumulated proportion of true-positives and true-negatives in the obtained 2×2 confusion matrix).

BBB impairment was defined as CSF-to-serum ratio of albumin greater than 9.0.

Statistical Analysis—Best Model

Least absolute shrinkage and selection operator (LASSO) logistic regression was used to select relevant biomarker features and build a predictive model of BBB impairment. All biomarker variables were included in the model, together with the variables used in the Reference model (age, gender, presence of ApoEe4 allele, CDR, education, CSF abeta₁₋₄₂, phospho-tau181 and total tau (t-tau)). These reference variables were included as non-penalised variables to ensure they were not filtered out by the LASSO selection process and to permit comparability with the reference model. A 10-fold cross-validation process was performed for each LASSO analysis using the glmnet package which permits estimation of the 95% confidence interval for the misclassification error for each value of the regularisation parameter. The LASSO analysis was repeated 100 times. The model that minimised the cross-validated misclassification error across the 100 runs was selected. Its performance was assessed by ROC area under the curve (AUC) estimation and compared with the Reference model.

Results and Discussion

Baseline characteristics are shown in Table 1. 118 subjects passed pre-analytical quality control for missing data, of which 13.5% (n=16) met the criteria for blood-brain barrier (BBB) impairment. There were no significant differences between age, education, MMSE, HAD scale, CDR, presence of ApoEe4 allele, CSF abeta₁₋₄₂, CSF t-tau and CSF phospho-tau181 between subjects with and without BBB impairment. However, there were more men with BBB impairment. Consistent with the literature, subjects with CDR 0.5/1 compared to CDR 0 had significantly higher albumin ratio verifying the functional significance of BBB function.

TABLE 1
Clinical and demographic characteristics of the older adult population¹

| | All | | BBB intact | | BBB impairment | |
|---|---|---|---|---|---|---|
| | (n = 118) | | (n = 102) | | (n = 16) | |
| Age, y, mean (SD) | 70.2 | (7.8) | 69.8 | (7.7) | 72.8 | (8.2) |
| Gender, n (%) of Males | 42 | (35.59%) | 32 | (31.37%) | 10 | (62.50%) |
| ApoE4 carrier, n (%) | 37 | (31.36%) | 32 | (31.37%) | 5 | (31.25%) |
| Education, y, mean (SD) | 12.4 | (2.6) | 12.5 | (2.7) | 11.9 | (2.1) |
| MMSE scale, mean (SD) | 26.9 | (3.1) | 27.3 | (2.9) | 24.8 | (3.4) |
| CDR = 0, n (%)² | 48/118 | (40.68%) | 45/102 | (44.12%) | 3/16 | (18.75%) |
| Diabetes, n (%) | 11 | (9.48%) | 9 | (9.00%) | 2 | (12.50%) |
| Hypertension, n (%) | 41 | (35.34%) | 35 | (35.00%) | 6 | (37.50%) |
| CSF Albumin ratio, mean (SD) | 6.1 | (2.4) | 5.4 | (1.5) | 10.7 | (1.5) |
| CSF abeta₁₋₄₂ (pg/mL), mean (SD) | 841.5 | (262.9) | 836.0 | (250.1) | 876.6 | (341.1) |
| CSF t-tau (pg/mL), mean (SD) | 369.5 | (280.1) | 356.2 | (268.9) | 454.4 | (340.5) |
| CSF phospho-tau (pg/mL), mean | 61.9 | (35.5) | 61.5 | (36.8) | 64.0 | (26.3) |
| (SD) | | | | | | |
| CSF sICAM-1 (pg/mL), mean (SD) | 2400.5 | (671.4) | 2259.4 | (577.5) | 3291.0 | (531.9) |
| CSF VEGF (pg/mL), mean (SD) | 4.2 | (1.0) | 4.1 | (0.9) | 5.1 | (0.9) |
| CSF IL-8 (pg/mL), mean (SD) | 41.8 | (14.8) | 40.7 | (14.7) | 49.1 | (13.7) |
| CSF SAA (pg/mL), mean (SD) | 1963.2 | (5800.1) | 1231.3 | (1382.3) | 6582.8 | (14867.7) |
| CSF MDC (pg/mL), mean (SD) | 33.3 | (21.6) | 32.5 | (22.1) | 38.0 | (17.5) |

¹Mean and standard deviation (SD) unless denotation states otherwise, MMSE, Mini Mental State Examination; HAD, Hospital Anxiety and Depression Scale; CDR, Clinical Dementia Rating; APOE4, apolipoprotein E epsilon 4;
²CDR scores include 0 (n = 48), 0.5 (n = 61) and 1 (n = 9)

CSF Biomarkers for Classification of BBB Impairment

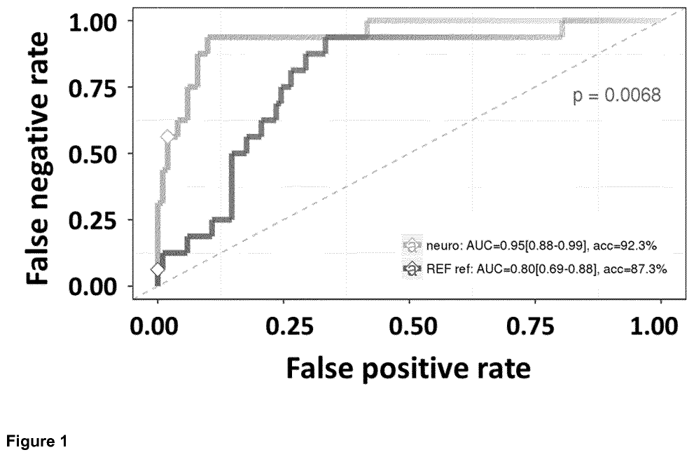

FIG. 1 illustrates the Reference and Best model ROC curves calculated for prediction of BBB impairment. The Reference model included age, gender, education, CDR, presence of ApoEe4 allele and CSF levels of abeta42, t-tau, and phospho-tau181 yielding a ROCAUC=0.80 and diagnostic accuracy for BBB impairment of 87.3%. The addition of the CSF neuroinflammatory biomarkers identified improved ROC AUC to 0.95 and the accuracy to 92.3%, with a Best model that included gender and 5 CSF biomarkers (sICAM-1, VEGF, IL-8, SAA, and MDC). The mean concentration differences between each of these 5 CSF biomarkers is illustrated in Table 1.

The five CSF biomarkers that best classify BBB impairment: CSF sICAM-1, VEGF, IL-8, SAA and MDC were all higher in individuals with BBB impairment.

Serum Biomarkers for Classification of BBB Impairment

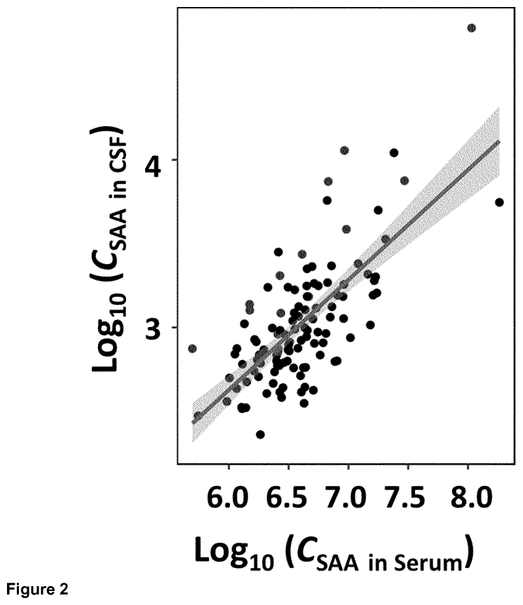

We observed a significant correlation in the concentrations of SAA measured both in CSF and serum in the cohort under study (FIG. 2). This observation suggested that, in humans, the determination of SAA concentration in serum might be a surrogate of SAA concentration in CSF. Sampling human serum and determining SAA concentrations in serum could offer a much less invasive alternative to SAA determinations in CSF.

All publications mentioned in the above specification are herein incorporated by reference. Various modifications and variations of the described methods of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention, which are obvious to those skilled in biochemistry and biotechnology or related fields, are intended to be within the scope of the following claims.

1. A method for determining whether a subject has an impaired blood-brain barrier (BBB) or is at risk of developing an impaired blood-brain barrier (BBB) comprising determining the level of one or more biomarkers in one or more samples obtained from the subject, wherein the one or more biomarkers comprise serum amyloid A (SAA).

2. A method for determining whether a subject is at risk of developing a cognitive impairment comprising determining the level of one or more biomarkers in one or more samples obtained from the subject, wherein the one or more biomarkers comprise serum amyloid A (SAA).

3. The method of claim 2, wherein the cognitive impairment is selected from the group consisting of Alzheimer's disease, vascular cognitive impairment and vascular dementia, Parkinson's disease, age-related cognitive decline and traumatic brain injury.

4. The method of claim 1, wherein the SAA is selected from the group consisting of SAA1, SAA2 and SAA4.

5. The method of claim 1, wherein the method further comprises determining the level of macrophage-derived chemokine (MDC) in a sample from the subject.

6. The method of claim 1, wherein the method further comprises determining the level of one or more biomarkers selected from the group consisting of soluble inter-cellular adhesion molecule-1 (sICAM-1), vascular endothelial growth factor (VEGF) and interleukin 8 (IL-8), in one or more samples obtained from the subject.

7. The method of claim 1, wherein the method comprises determining the level of SAA, MDC, sICAM-1, VEGF and IL-8 in one or more samples obtained from the subject.

8. The method of claim 1, wherein the method comprises determining a blood-brain barrier (BBB) impairment score (S) using the formula:
S=A+B×(IL-8)+C×(MDC)+D×(SAA)+E×(sICAM-1)+F×(VEGF)+G×(Gender)
wherein A, B, C, D, E, F and G are coefficients.

9. The method of claim 1, wherein the levels of SAA, MDC, sICAM-1, VEGF and/or IL-8 are determined in one or more cerebrospinal fluid and/or serum samples.

10. The method of claim 1, wherein the subject is a human over the age of 55 years old.

11. A method of treating or preventing blood-brain barrier (BBB) impairment comprising the steps:
(a) determining whether a subject has an impaired blood-brain barrier (BBB) or is at risk of developing an impaired blood-brain barrier (BBB) comprising determining the level of one or more biomarkers in one or more samples obtained from the subject, wherein the one or more biomarkers comprise serum amyloid A (SAA); and
(b) applying an intervention capable of improving blood-brain barrier (BBB) function to a subject identified to be in need thereof.

12. (canceled)

13. The method of claim 11, wherein the intervention is a dietary intervention.

14. The method of claim 13, wherein the dietary intervention comprises increasing vitamin B intake by the subject.

15. The method of claim 13, wherein the dietary intervention comprises increasing omega-3 fatty acid intake by the subject.

16. The method of claim 2, wherein the SAA is selected from the group consisting of SAA1, SAA2 and SAA4.

17. The method of claim 2, wherein the method further comprises determining the level of macrophage-derived chemokine (MDC) in a sample from the subject.

18. The method of claim 2, wherein the method further comprises determining the level of one or more biomarkers selected from the group consisting of soluble inter-cellular adhesion molecule-1 (sICAM-1), vascular endothelial growth factor (VEGF) and interleukin 8 (IL-8), in one or more samples obtained from the subject.

19. The method of claim 2, wherein the method comprises determining the level of SAA, MDC, sICAM-1, VEGF and IL-8 in one or more samples obtained from the subject.

20. The method of claim 2, wherein the method comprises determining a blood-brain barrier (BBB) impairment score (S) using the formula:
S=A+B×(IL-8)+C×(MDC)+D×(SAA)+E×(sICAM-1)+F×(VEGF)+G×(Gender)
wherein A, B, C, D, E, F and G are coefficients.

21. The method of claim 2, wherein the levels of SAA, MDC, sICAM-1, VEGF and/or IL-8 are determined in one or more cerebrospinal fluid and/or serum samples.
